(19) United States
(12) Patent Application Publication
(10) Pub. No.: US 2026/0283194 A1
(43) Pub. Date: Sep. 24, 2026

(54) BACILLUS COMPOSITION TO IMPROVE GASTROINTESTINAL HEALTH AND FAECAL SCORE

(71) Applicant: CHR. HANSEN A/S, Hoersholm (DK)

(72) Inventors: Lidiia ALAVERDOVA, Hoersholm (DK); Dorthe SANDVANG, Slangerup (DK); Erik BOLL, Hoersholm (DK)

(73) Assignee: CHR. HANSEN A/S, Hoersholm (DK)

(21) Appl. No.: 19/469,194

(22) PCT Filed: Mar. 25, 2024
(86) PCT No.: PCT/EP2024/057989

§ 371 Date: Sep. 25, 2025

(30) Foreign Application Priority Data
Mar. 30, 2023 (EP) 23165579.6

(51) Int. Cl. A23K 10/18; A23K 50/40; C12N 1/205; C12R 1/125

(52) U.S. Cl. CPC A23K 10/18; A23K 50/40; C12N 1/205; C12R 2001/125

(57) ABSTRACT

The present disclosure generally relates to compositions and methods for improving the faecal score of an animal using a novel combination of probiotic Bacillus strains and administering said composition to the animal. The disclosure also relates to compositions and methods for improving gastrointestinal health of an animal using a novel combination of probiotic Bacillus strains. The disclosure further relates to compositions and methods of increasing release of sugars and protein solubility of an animal feed using a combination of probiotic Bacillus strains.

### FIELD OF THE INVENTION

The present disclosure generally relates to compositions and methods for improving the faecal score using a novel combination of probiotic Bacillus strains and administering said composition to the animal. Also, the disclosure relates to compositions and methods for improving gastrointestinal health using a novel combination of probiotic Bacillus strains. The disclosure also relates to compositions and methods of increasing release of sugars and protein solubility of an animal feed, using a novel combination of probiotic Bacillus strains.

### BACKGROUND

Bacillus based probiotic feed additives are known for their positive effects on health in animal feed. These products are relevant for the feed industry because spores are heat stable and can survive the pelletizing process at temperatures up to 90-95° C. The endospore-forming bacteria Bacillus subtilis and Bacillus amyloliquefaciens are Generally Regarded as Safe (GRAS) by the U.S. Food and Drug Administration (FDA) and acceptable for inclusion in an animal diet or water by the Association of American Feed Control Officials (AAFCO).

Many pet owners are concerned about the general health of their animals, including the gastrointestinal health. Especially dog owners are interested in improving the overall health of the gastrointestinal system of their pet including improving the faecal score/the faecal consistency of the dog. Attempts exist in the prior art of providing solutions which improve the faecal score, however further improvements are still needed in the field to achieve the practical benefits for the pet owner. It would be highly advantageous to provide a solution which was easy to administer by the pet owner and which improved the gastrointestinal health and the faecal consistency of the pet in a significant way. If the digestibility of the animal feed provided to the pet can also be improved, that would be an added benefit.

### SUMMARY OF THE INVENTION

The present disclosure provides a Bacillus strain selected from the group consisting of a) the strain deposited as DSM32324, b) the strain deposited as DSM32325, and c) the strain deposited as DSM 25840, which has sensitivity for ampicillin, vancomycin, gentamicin, kanamycin, streptomycin, erythromycin, clindamycin, tetracycline, and chloramphenicol; and has inhibitory activity against E. coli and Clostridium perfringens. Thus, the strains of the present disclosure have all been considered susceptible to antibiotics as specified in EFSA Journal 2012; 10(6):2740.

Example 1 provides the result of an in vitro reducing sugar release assay. The composition of the present disclosure provides an improvement in carbohydrate degradation and release of sugars from an animal feed.

Example 2 provides the result of an in vitro protein solubility assay. The composition of the present disclosure provides an increase in protein solubility. While not wishing to be bound by theory, it is believed that the composition of the present disclosure may improve the utilization of the animal feed by increasing both energy and protein release from the animal feed. This is believed to result in increased uptake of energy and protein by the animal.

The composition of the present disclosure may provide an improvement in the gastrointestinal health of the animal as well as an improvement of the faecal score of the animal receiving the composition.

Example 3 provides the result of an in vivo trial showing an improvement in faecal score amongst dogs receiving the composition of the present disclosure.

While not wishing to be bound by theory, it is believed that providing an animal with the composition of the present disclosure may improve the overall gastrointestinal health of the animal and may improve the faecal score achieved while at the same time it may improve digestibility of the animal feed and lead to increased release of sugars and increased protein solubility.

Further, it is contemplated that by increasing protein uptake, less protein may reach the animal's hindgut for microbial fermentation, thereby decreasing the production of putrefactive compounds and decreasing faecal malodor.

### Definitions

In general, the terms and phrases used herein have their art-recognized meaning, which can be found by reference to standard textbooks, journal references and context known to those skilled in the art. The following definitions are provided to clarify their specific use in context of the present disclosure.

As used herein, the singular forms “a”, “an” and “the” are intended to include the plural forms as well, unless the context clearly indicates otherwise.

As used herein, the term “and/or” is intended to mean the combined (“and”) and the exclusive (“or”) use, i.e. “A and/or B” is intended to mean “A alone, or B alone, or A and B together”.

Animal: The term “animal” includes, but is not limited to canine (e.g., dogs), feline (e.g., cats); equine (e.g., horses), bovine (e.g., cattle), ovine (e.g. sheep), caprine (e.g. goat) porcine animals (e.g. swine, such as piglets, weaners, growers, finishers, hocks, polts, gilts, sows, gestation sows) and rabbits, as well as avians including, but not limited to, poultry such as broilers, breeders, layers, ostriches, quails, turkeys, ducks, geese, domestic fur animals such as ferrets, minks, mustelids, ruminants such as cattle, sheep, goats, camels, and giraffes, and fish such as fin-fish, shellfish, and other aquatic animals. Fin-fish include all vertebrate fish, which may be bony or cartilaginous fish, such as salmon, trout, tilapia, catfish and carps, and shellfish include, but are not limited to clams, lobster, shrimp, crab, prawns and oysters.

As used herein, the term “animal feed” refers to any compound, preparation, or mixture suitable for, or intended for intake by an animal. Animal feed may comprise concentrates as well as for example vitamins, minerals, enzymes, amino acids and/or other feed ingredients (such as in a premix).

As used herein, the term “complete feed” is often used for animal feed for pets such as dogs and cats and refers to a ready-to-eat animal feed which comprises concentrates as well as for example vitamins, minerals, enzymes, amino acids and/or other feed ingredients (such as in a premix). An example of a complete feed for a dog is the control diet given in Example 2.

As used herein, the term “composition” refers to a composition comprising a carrier and at least one bacterial strain as described herein. The compositions described herein may be a probiotic, a Direct Fed Microbial (DFM), an animal feed additive or premix, or an animal feed such as complete feed.

As used herein, the term “direct fed microbial” or “DFM” means live micro-organisms including spores which, when administered in adequate amounts, confer a benefit, such as improved gastrointestinal digestion or health, on the host.

As used herein the term “Dry Matter Content” means the amount of dry matter in faeces after transition through the gastrointestinal tract of the animal, excluding water content.

As used herein, the terms “effective amount”, “effective concentration”, or “effective dosage” are defined as the amount, concentration, or dosage of the bacterial strain(s) sufficient to improve the wellness, health, digestion or yield of an animal. The actual effective dosage in absolute numbers depends on factors including the state of health of the animal in question. The “effective amount”, “effective concentration”, or “effective dosage” of the bacterial strains may be determined by routine assays known to those skilled in the art. Examples of effective amounts are described in Example 2.

As used herein, the terms “feeding an animal” or “fed to an animal” means that the composition of the present invention is administered orally to the animal in an effective amount. The oral administration may be repeated, e.g. one or more times daily over a specified time period such as several days, one week, several weeks, one months or several months. Accordingly, the terms “feeding” or “fed” mean any type of oral administration such as administration via an animal feed or via drinking water or, in certain circumstances, by oral gavage or aerosol spray.

As used herein, the term “faecal score” or “faecal consistency” are used interchangeable and refers to the scoring scheme described in Example 2. An improved faecal score according to the present disclosure is an average faecal score above 3 as measured over a period of time.

As used herein, the term “isolated” means that the bacterial strains described herein are in a form or environment which does not occur in nature, i.e. the strain is at least partially removed from one or more or all of the naturally occurring constituents with which it is associated in nature.

As used herein the term “carbohydrates” means refers to saccharides which is a group of molecules that includes sugars, starch, and fiber. The saccharides are divided into four chemical groups of varying size: monosaccharides (e.g. glucose), disaccharides, oligosaccharides, and polysaccharides. Along with proteins and fats, carbohydrates are one of three main nutrients found in animal feed. The body of the animal breaks down carbohydrates into glucose. Glucose, or blood sugar, is the main source of energy for the body's cells, tissues, and organs.

As used herein, the term “pet” refers to companion animals such as domestic cat and domestic dog, ferrets, rats, rabbit, hamster, guinea pig, reptiles, birds such as parakite, parrot, falcon, hawk. In one embodiment of the disclosure the pet is a dog.

As used herein, the term “pet food” includes a dry, semi-moist or a moist (wet) product. Wet food includes food that has a moisture content of 70%-90% and is usually sold in containers such as tins, pouches and/or trays. Dry food includes food having a similar composition but with 5%-15% moisture, often presented as small biscuit-like kibbles. Semi-moist food includes food having a moisture content of from above 15% up to 70%. The amount of moisture in any product may influence the type of probiotic bacteria that can be added. The food product, of any moisture level, may be ready to eat. Pet food snack includes snack bars, cereal bars, snacks, biscuits, drinks (e.g. cat milk) and sweet products.

The pet food product may be a cooked product, it may incorporate meat or animal derived material (such as beef, chicken, eggs, turkey, lamb, fish, blood marrow, marrowbone, etc. or by-products hereof). Alternatively, the composition may be meat-free (preferably including a meat substitute such as soya, maize, gluten or a soya product) in order to provide protein. The composition may contain additional protein sources such as soya protein concentrate, milk, protein, gluten etc. The composition may also include starch, such as one or more grains (e.g. wheat, corn, rice, oats, barley etc.) or may be starch free. The composition may incorporate or may be a gelatinised starch matrix. The composition may incorporate one or more types of fibre such as sugar beet pulp, chicory pulp, chicory, coconut endosperm fibre, wheat fibre, fructooligosaccharides, galactooligosaccharides, inulin etc. The fibre may be a prebiotic. The combination of the probiotic and the prebiotic may form a synbiotic combination. The most suitable composition is pet food described herein which is sold as a pet food and in particular a pet food for a domestic dog or a domestic cat. Preferably, the pet food is in a dry format, such as a dried ready-to-eat cereal product comprising a cooked starch matrix produced by extrusion cooking and referred to as pet kibble.

As used herein, the term “premix” refers to a composition comprising at least two strains added to a carrier to make a premix which is then added to an animal feed at a desired inclusion rate.

As used herein, the term “probiotics” are often described as dietary supplements containing potentially beneficial bacteria and according to the currently adopted definition are live micro-organisms which can confer a health benefit on the host. Probiotic micro-organisms assist the body's naturally occurring gut flora to re-establish themselves. Probiotic micro-organisms are reported and known to strengthen the immune system and prevent infections, reduce inflammation, improve mineral absorption and prevent the growth of harmful bacteria. Probiotics are in some parts of the world called Direct Fed Microbials (DFM's).

As used herein, the term “protein” refers to any compound, preparation or mixture that includes at least one protein derived from or originating from a vegetable or animal source, including modified proteins and protein-derivatives. Vegetable proteins may be derived from vegetable protein sources, such as legumes and cereals, for example materials from plants of the families Fabaceae (Leguminosae), e.g. soybean, lupine, pea, or bean; Cruciferaceae, Chenopodiaceae, e.g. beet, sugar beet, spinach or quinoa; and Poaceae. Other examples of vegetable protein sources are cereals such as barley, wheat, rye, oat, maize (corn), rice, and sorghum. Animal proteins are derived from animal protein sources such as meat sources e.g. poultry, lamb, pork, fish or animal by-products e.g. fish and bone meal.

Proteins are complex molecules present in all living organisms and provide many essential functions in the body. These molecules are formed through the ‘building blocks’ of 20 amino acids, which can be deemed essential or non-essential—those deemed essential cannot be synthesized in the body and must be obtained through dietary intake, whereas non-essential can be synthesized by the body through the metabolism of other amino acids. The number of essential amino acids depends on the species in question. Solubilization of protein is thus needed for supplying essential amino acids to build hair, skin, nails, muscles, tendons, ligaments, and cartilage. Protein also plays a main role in hormone production and promotes immune system function.

As used herein, the term “reducing sugar” means any sugar that either has a reactive aldehyde group or is capable of forming one to allow the sugar to act as a reducing agent (also known as simple sugars). The reducing ends are formed by the enzymatic cleavage of the glycosidic bond between polymeric carbohydrates. Reducing sugars include glucose, glyceraldehyde and galactose as well as disaccharides, like lactose and maltose and can be measured by the Nelson-Somogyi (NS) or dinitrosalicylic acid (DNS) method. DNS is an aromatic compound that reacts with reducing sugars and other reducing molecules to form 3-amino-5-nitrosalicylic acid, which absorbs light strongly at 540 nm. The assay simulates the situation when feed is ingested by the animal and is digested in the digestive tract. The ability of the composition of the present disclosure comprising Bacillus strains to degrade carbohydrate to release sugars has been investigated in Example 1.

As used herein, the term “susceptible to antibiotics” means the phenotypic property of a bacterial strain, that growth of said bacterial strain is inhibited under conditions where the bacterial strain would otherwise grow. In this context susceptibility to antibiotics is tested after the CLSI guidelines (M07-A8 and M45-A2). A strain of Bacillus is considered susceptible if growth is only detected at or below the breakpoint concentration specified in EFSA Journal 2012; 10(6):2740 for vancomycin, gentamicin, kanamycin, streptomycin, erythromycin, clindamycin, tetracycline, and chloramphenicol. With regard to ampicillin there is no breakpoint given by EFSA for Bacillus; the breakpoint 4 mg/L has been chosen for a strain to be considered susceptible.

As used herein, the terms “spore” and “endospore” are interchangeable and have their normal meaning which is well known and understood by those of skill in the art. As used herein, the term spore refers to a microorganism in its dormant, protected non-reproductive state.

### BRIEF DESCRIPTION OF THE FIGURES

FIG. 1 shows the result of an in vitro assay examining release of sugars (labeled as “glucose equivalents”) after animal feed samples are mixed in liquid media with the composition of the disclosure (probiotics sample denoted “PRO”) and without the composition of the invention (control sample denoted “Control”).

FIG. 2 shows the result of an in vitro assay investigating the solubility of protein after animal feed samples are mixed in liquid media with the composition of the disclosure (probiotics sample denoted “PRO”) and without the composition of the invention (control sample denoted “Control”).

### DETAILED DESCRIPTION OF THE INVENTION

This disclosure relates to an animal feed, animal feed additive, or animal feed premix composition comprising at least two Bacillus strains selected from the group consisting of the strain deposited as DSM 32324, DSM 32325, and DSM 25840, which
(i) are susceptible to ampicillin, vancomycin, gentamicin, kanamycin, streptomycin, erythromycin, clindamycin, tetracycline, and chloramphenicol;
(ii) have inhibitory activity against E. coli, Salmonella and Clostridium perfringens,
(iii) have the ability to facilitate degradation of carbohydrates and thereby increase the amount of available sugars in animal feed,
(iv) have the ability to facilitate protein solubility and thereby increase the amount of solubilized protein in animal feed and
(iiii) have the ability to improve the gastrointestinal health and the average faecal score in the animal.

The bacterial strains described herein are isolated, i.e. present in a form or environment which does not occur in nature.

A Bacillus strain of the present disclosure is considered to exhibit an inhibitory activity towards E. coli if the inhibition zone is at least 0.5 mm (low inhibition). Preferably, the inhibition zone is at between at least 0.5 mm and 2 mm (medium), more preferably more than 2 mm (high). The inhibition zone may be different for the various E. coli strains. For a strain to be considered to exhibit an inhibitory activity against E. coli according to the present invention it should exhibit an inhibition zone of at least 0.5 mm for all of the E. coli strains tested. Preferably, the inhibition zone of two, three, four or even more preferably the inhibition zone of all five of the E. coli strains is at least between 0.5 mm and 2 mm. Even more preferably, the inhibition zone of two, three, four or even more preferably the inhibition zone of all five of the E. coli strains is more than 2 mm.

A Bacillus strain of the present disclosure is considered to exhibit an inhibitory activity towards Clostridium perfringens if the inhibition zone is at least 0.5 mm (low inhibition). Preferably, the inhibition zone is at between at least 0.5 mm and 2 mm (medium), more preferably more than 2 mm (high). The inhibition zone may be different for the various Clostridium perfringens strains. For a strain to be considered to exhibit an inhibitory activity against Clostridium perfringens according to the present invention it should exhibit an inhibition zone of at least 0.5 mm for all of the Clostridium perfringens strains tested. Preferably, the inhibition zone of two, three, four or even more preferably the inhibition zone of all five of the Clostridium perfringens strains is at least between 0.5 mm and 2 mm. Even more preferably, the inhibition zone of two, three, four or even more preferably the inhibition zone of all five of the Clostridium perfringens strains is more than 2 mm.

A Bacillus strain of the present disclosure is considered to exhibit an inhibitory activity towards Salmonella if the inhibition zone is at least 0.5 mm (low inhibition). Preferably, the inhibition zone is at between at least 0.5 mm and 2 mm (medium), more preferably more than 2 mm (high). The inhibition zone may be different for the various Salmonella strains. For a strain to be considered to exhibit an inhibitory activity against Salmonella according to the present invention it should exhibit an inhibition zone of at least 0.5 mm for all of the Salmonella strains tested. Preferably, the inhibition zone of two, three, four or even more preferably the inhibition zone of all five of the Salmonella strains is at least between 0.5 mm and 2 mm. Even more preferably, the inhibition zone of two, three, four or even more preferably the inhibition zone of all five of the Salmonella strains is more than 2 mm.

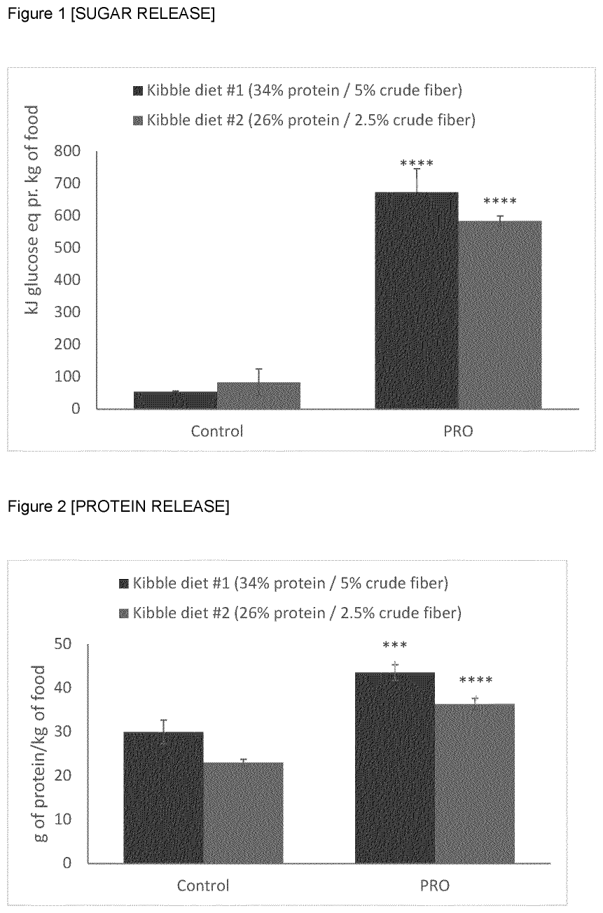

Preferably, a Bacillus strain of the present disclosure should also be able to increase the amount of sugars from degradation of crude fiber and/or starch components of animal feed. The ability of the Bacillus strains in the composition of the present disclosure to degrade carbohydrates to sugars has been investigated in Example 1 and the results are provided in FIG. 1. Bacillus strains having the ability to increase the available sugars amount to at least 500 kJ/kg feed when tested as outlined in the example are considered preferable.

The person of ordinary skill in the art is able to repeat these assays to determine whether a specific Bacillus strain complies with the antibiotic sensitivity, the inhibitory activity and the capability of degrading carbohydrates and proteins. In this manner the person of ordinary skill in the art will be able to consistently test strains for the stated properties. Evidently, these assays can be performed in any order and some strains may be excluded during the process if they do not fulfill the criteria.

The disclosure further provides a Bacillus composition comprising from 10⁵ to 10¹² colony forming units per gram (CFU/g) of at least two Bacillus strains of the disclosure. In one aspect of the disclosure, the Bacillus composition comprises two Bacillus strains of the disclosure, for example a combination of Bacillus subtilis DSM 32324 and Bacillus amyloliquefaciens DSM 25840.

In another aspect of the disclosure the Bacillus composition comprises three Bacillus strains of the disclosure, for example a combination of the Bacillus subtilis strain deposited at DSMZ under accession number DSM 32324, the Bacillus amyloliquefaciens strain deposited at DSMZ under accession number DSM 25840 and the Bacillus subtilis strain deposited at DSMZ under accession number DSM 32325.

The Bacillus compositions according to the invention may comprise a combination of at least two of the Bacillus strains of the invention and at least one other Bacillus strain. The Bacillus composition may comprise at least two strains, such as at least three, such as at least four, such as at least five Bacillus strains, at least two of which are a Bacillus strain of the present invention.

Bacillus strains may be used in any combination in the Bacillus compositions. For example, the Bacillus composition may comprise at least one Bacillus strain of the invention and/or at least one Bacillus licheniformis strain and/or at least one Bacillus amyloliquiefaciens strain e.g. two Bacillus subtilis strains of the invention and at least one Bacillus amyloliquiefaciens strain.

The composition may comprise Bacillus subtilis DSM 32324 and/or Bacillus subtilis DSM 32325 in combination with Bacillus amyloliquiefaciens DSM 25840 and/or Bacillus licheniformis DSM 17236 and/or Bacillus subtilis DSM 19489 and/or Bacillus subtilis DSM 17231. Any other possible combination of the Bacillus strains of the present invention with other Bacillus strains may also be made.

As a specific example, the Bacillus composition comprises Bacillus subtilis DSM 32324, Bacillus subtilis DSM 32325 and Bacillus amyloliquiefaciens DSM 25840. As another specific example, the Bacillus composition comprises Bacillus subtilis DSM 32324, Bacillus subtilis DSM 32325 and Bacillus licheniformis DSM 17236. In a yet further specific example, the Bacillus composition comprises Bacillus subtilis DSM32324, Bacillus subtilis DSM32325 and Bacillus subtilis DSM19489.

In a particular embodiment, the composition further comprises a bacterium from one or more of the following strains of Bacillus: Bacillus amyloliquefaciens, Bacillus subtilis, Bacillus pumilus, Bacillus polymyxa, Bacillus licheniformis, Bacillus megaterium, Bacillus coagulans, Bacillus circulans, Bacillus simplex, Bacillus mojavensis, Bacillus safensis, Bacillus simplex, Bacillus atrophaeus, Bacillus methylotrophicus, Bacillus siamensis, Bacillus vallismortis, Bacillus tequilensis or any combination thereof.

In a further embodiment, the composition comprises a bacterium from one or more of the following genera: Lactobacillus, Lactococcus, Streptococcus, Bacillus, Pediococcus, Enterococcus, Leuconostoc, Carnobacterium, Propionibacterium, Bifidobacterium, Clostridium and Megasphaera or any combination thereof.

Preferred examples of such bacteria are the species Enterococcus faecium, such as the Enterococcus faecium DSM 22502.

As a specific example, the Bacillus composition comprises Bacillus subtilis DSM 32324, Bacillus amyloliquiefaciens DSM 25840 and Enterococcus faecium DSM 22502.

In a particular embodiment, the composition further comprises one or more types of yeast. The one or more types of yeast can be selected from the group consisting of Saccharomycetaceae, Saccharomyces (such as S. cerevisiae and/or S. boulardii), Kluyveromyces (such as K. marxianus and K. lactis), Candida (such as C. utilis, also called Torula yeast), Pichia (such as P. pastoris), Torulaspora (such as T. delbrueckii), Phaffia yeasts and Basidiomycota.

It is contemplated that the proportion of each strain in the composition will be 1 to 99%, such as 20 to 80%, e.g. 30 to 70%, more particularly 20%, 33%, 40% or 50% of the total amount of bacterial isolates calculated as CFU/g composition. The individual strains may be present in about equal numbers or in unequal numbers.

The relevant strain or strains are provided in a commercially relevant form known to the skilled person. Accordingly, in an embodiment the strain or strains of the composition are present in a dried (e.g. spray dried) or frozen form. The composition may be provided in any suitable form such as in the form of a liquid e.g. a gel, a slurry, a powder or a pellet. In a preferred aspect of the disclosure, the composition is in the form of a dry powder, a capsule or an oral paste.

In one embodiment, the composition comprises from 10⁵ to 10¹² CFU/g, more preferably from 10⁶ to 10¹² CFU/g, and most preferably from 10⁷ to 10¹² CFU/g, such as from 10⁸ to 10¹¹ CFU/g, e.g. from 10⁹ to 10¹⁰ CFU/g of each of the bacterial strains in the composition (i.e. the Bacillus strains disclosed herein). The composition comprises at least 5×10⁴ CFU of each strain per gram of the composition which distinguishes a composition of the present invention from e.g. animal feed with naturally occurring strains.

The term “CFU/g” relates to the gram weight of the composition including carriers such as calcium carbonate, anti-caking agents such as aluminium silicates and kieselgur (diatomaceous earth), and other components present in the composition.

Compositions of the present invention include at least two Bacillus strains of the disclosure and at least one carrier, excipient and/or diluent and/or other component that make the composition suitable for feeding to an animal or as an additive for drinking water. Where administered to an animal, it will be non-toxic to the animal. There is a myriad of such agents available which may be added. Without intending to be limiting, examples include standardizing agents, extenders, wetting agents and lubricating agents, preservative agents, lipids, stabilizers, solubilizers, free flowing agents, and emulsifiers. Examples that may be particularly useful in administration to an animal include ground corn cobs, salt, ground limestone, calcium carbonate, sodium bentonite, zeolites, ground soy hulls, citrus pulp, dairy by-products, animal protein products, grain products, plant protein products, processed grain products and by-products, roughage products, molasses products fermentation by-products such as dried distillers grains and/or solubles, citric acid and glutamic acid fermentation by-products and the like.

Bacillus bacteria exist as spores and vegetative cells which can divide to produce more vegetative cells. When reference is made herein to Bacillus, this relates to both spores and vegetative cells unless the context indicates otherwise.

In the composition of the present disclosure, the Bacillus strains are preferably provided as spores. The primary function of sporulation is generally to ensure the survival of a bacterium through periods of environmental stress. They are therefore resistant to ultraviolet and gamma radiation, desiccation, lysozyme, temperature, starvation, and chemical disinfectants. The spore coat is impermeable to many toxic molecules and may also contain enzymes that are involved in germination. The core has normal cell structures, such as DNA and ribosomes, but the spore is metabolically inactive.

In one aspect, the present invention provides an animal feed, animal feed additive or premix comprising at least two Bacillus strains according to the disclosure, and further comprising one or more of concentrate(s), vitamin(s), mineral(s), enzyme(s), amino acid(s) and/or other feed ingredient(s), such as flavours, yeast extract, active charcoal, mineral clay, silica or a combination thereof.

Animal feed for carnivores and omnivores, e.g. pets such as cats and dogs, generally comprising a starch source, texturized proteins, or semi-moist pieces can be produced using typical ingredients used to manufacture pet food such as grain sources (e.g., com, rice, wheat, bailey), protein sources (e.g., meat sources such as poultry, beef, lamb, pork, fish) or animal by-products e.g. fish and bone meal, vegetable sources (e.g., soy, corn gluten, casein, whey, eggs); fats (e.g., vegetable oils, animal fats, fish oils), plant fibres (beet pulp, soy hulls, cellulose) optionally vitamins (e.g., Vitamin E, C, BI, B2, B6); and minerals (e.g., calcium sources, phosphorus sources, salts, trace minerals) and various flavorants or palatants, processing aids, and preservatives to make a pet food that meets a pet's nutritional requirements and possesses the necessary aesthetic characteristics.

In a particular embodiment, the animal feed consists of or comprises milk (e.g. from sow, cow, goat, sheep), e.g. for feeding of neonatal animals. In another particular embodiment, the animal feed consists of or comprises milk replacement, e.g. for feeding of neonatal animals.

In another embodiment, the animal feed may include one or more vitamins, such as one or more fat-soluble vitamins and/or one or more water-soluble vitamins. In another embodiment, the animal feed may optionally include one or more minerals, such as one or more trace minerals and/or one or more macro minerals. Usually fat- and water-soluble vitamins, as well as trace minerals form part of a so-called premix intended for addition to the feed, whereas macro minerals are usually separately added to the feed. Non-limiting examples of fat-soluble vitamins include vitamin A, vitamin D3, vitamin E, and vitamin K, e.g. vitamin K3. Non-limiting examples of water-soluble vitamins include vitamin B12, biotin and choline, vitamin 1, vitamin B2, vitamin B6, niacin, folic acid and panthothenate, e.g. Ca-D-panthothenate. Non-limiting examples of trace minerals include boron, cobalt, chloride, chromium, copper, fluoride, iodine, iron, manganese, molybdenum, selenium and zinc. Non-limiting examples of macro minerals include calcium, magnesium, potassium and sodium.

The animal feed, animal feed additive or premix of the invention may also comprise at least one enzyme selected from the group comprising of phytase (EC 3.1.3.8 or 3.1.3.26); xylanase (EC 3.2.1.8); galactanase (EC 3.2.1.89); alpha-galactosidase (EC 3.2.1.22); protease (EC 3.4); phospholipase A1 (EC 3.1.1.32); phospholipase A2 (EC 3.1.1.4); lysophospholipase (EC 3.1.1.5); phospholipase C (3.1.4.3); phospholipase D (EC 3.1.4.4); amylase such as, for example, alpha-amylase (EC 3.2.1.1); lysozyme (EC 3.2.1.17); and beta-glucanase (EC 3.2.1.4 or EC 3.2.1.6), or any mixture thereof.

The animal feed such as the complete feed, animal feed additive or premix of the invention may further comprise one or more added amino acids. Examples of amino acids which are used in animal feed are lysine, alanine, beta-alanine, threonine, methionine and tryptophan. The animal feed, animal feed additive or premix of the invention may further comprise colouring agents, stabilisers, growth improving additives and aroma compounds/flavourings, polyunsaturated fatty acids (PUFAs); reactive oxygen generating species, anti-microbial peptides and anti-fungal polypeptides. Examples of colouring agents are carotenoids such as beta-carotene, astaxanthin and lutein. Examples of aroma compounds/flavorings are creosol, anethol, deca-, undeca- and/or dodeca-lactones, ionones, irone, gingerol, piperidine, propylidene phatalide, butylidene phatalide, capsaicin and tannin. Examples of polyunsaturated fatty acids are C18, C20 and C22 polyunsaturated fatty acids, such as arachidonic acid, docosohexaenoic acid, eicosapentaenoic acid and gamma-linoleic acid. Examples of reactive oxygen generating species are chemicals such as perborate, persulphate, or percarbonate; and enzymes such as an oxidase, an oxygenase or a syntethase.

In one embodiment, the animal feed, such as the complete feed, animal feed additive or premix comprises one or more coccidiostats.

The animal feed such as the complete feed, animal feed additive or premix further comprises a carrier. The carrier can comprise one or more of the following compounds: water, glycerol, ethylene glycol, 1,2-propylene glycol or 1,3-propylene glycol, sodium chloride, sodium benzoate, potassium sorbate, sodium sulfate, potassium sulfate, magnesium sulfate, sodium thiosulfate, calcium carbonate, sodium citrate, dextrin, maltodextrin, glucose, sucrose, sorbitol, lactose, whey, whey permeate, wheat flour, wheat bran, corn gluten meal, starch and cellulose.

In one embodiment, the one or more bacterial strains are stable when subjected to pressures applied/achieved during an extrusion process for pelleting. In a particular embodiment, the one or more bacterial strains are stable at pressures ranging from 1 bar to 40 bar.

In a particular embodiment, the one or more bacterial strains are stable at high temperatures. In particular, the bacterial strains are stable when they are subjected to temperatures achieved during an extrusion process for pelleting. In an even more particular embodiment, the one or more bacterial strains are stable at temperatures ranging from 70° C. to 120° C.

Animal diets can e.g. be manufactured as mash feed (non-pelleted), pelleted feed, or extruded feed. Typically, the milled feed-stuffs are mixed and sufficient amounts of essential vitamins and minerals are added according to the specifications for the species in question. The bacteria cultures and optionally enzymes can be added as solid or liquid formulations. For example, for mash feed a solid or liquid culture formulation may be added before or during the ingredient mixing step. For pelleted feed the (liquid or solid) Bacillus composition may also be added before or during the feed ingredient step. Typically a liquid Bacillus composition of the invention comprises the bacterial strain(s) optionally with a polyol, such as glycerol, ethylene glycol or propylene glycol, and is added after the pelleting or extrusion step, such as by spraying the liquid formulation onto the pellets or kibbles. The bacteria may also be incorporated in an animal feed additive or premix.

Another aspect of the disclosure relates to a method for feeding an animal comprising administering a composition of the disclosure to an animal, in particular a companion animal or pet, such as a dog or cat.

In another aspect, the disclosure relates to the use of at least two Bacillus strains of the disclosure or an animal feed, animal feed additive or premix comprising at least two Bacillus strains of the disclosure to improve the gastrointestinal health and faecal score of the animal, in particular a companion animal or pet, such as a dog or cat.

In another aspect, the disclosure relates to administration of a composition of the disclosure which increases reducing sugar release and protein solubility of an animal feed by adding the at least two Bacillus strains of the disclosure to an animal feed.

### DEPOSIT AND EXPERT SOLUTION

The applicant requests that a sample of the deposited microorganisms stated below may only be made available to an expert, subject to available provisions governed by Industrial Property Offices of States Party to the Budapest Treaty, until the date on which the patent is granted.

TABLE 1
The applicant has made the following deposits at
a Depositary institution having acquired
the status of international depositary authority
under the Budapest Treaty on the International
Recognition of the Deposit of Microorganisms
for the Purposes of Patent Procedure: Leibniz
Institute DSMZ-German Collection of
Microorganisms and Cell Cultures Inhoffenstr.
7B, 38124 Braunschweig, Germany.

| | | Deposit date |
|---|---|---|
| Strain | Accession No. | YYYY.MM.DD |
| Bacillus amyloliquefaciens | DSM 25840 | 2012 Apr. 3 |
| Bacillus subtilis | DSM 32324 | 2016 Jun. 8 |
| Bacillus subtilis | DSM 32325 | 2016 Jun. 8 |

### Embodiments

The invention is further defined by the following numbered paragraphs:
1. Use of an animal feed, animal feed additive, or animal feed premix composition comprising:
   a. from 10⁵ to 10¹² colony forming units per gram (CFU/g) of at least two Bacillus strains selected from:
      i. the Bacillus subtilis strain deposited at Deutsche Sammlung von Microorganismen und Zellkulturen GmbH (DSMZ) under accession number DSM 32324,
      ii. the Bacillus subtilis strain deposited at DSMZ under accession number DSM 32325, and
      iii. the Bacillus amyloliquefaciens strain deposited at DSMZ under accession number DSM 25840; and
   b. one or more animal feed, animal feed additive, or animal feed premix ingredients, for improving the gastrointestinal health and/or faecal score in an animal receiving the animal feed, animal feed additive, or animal feed premix.
2. Use according to paragraph 1, wherein the animal is a pet such as a dog or cat, preferably a dog.
3. Use according to paragraph 2, for improving the faecal score of a dog.
4. Use according to any preceding paragraph, wherein the at least two Bacillus strains comprise a Bacillus subtilis strain and a Bacillus amyloliquefaciens strain.
5. Use according to any preceding paragraph, wherein the at least two Bacillus strains comprise the Bacillus subtilis strain deposited at DSMZ under accession number DSM 32324 and the Bacillus amyloliquefaciens strain deposited at DSMZ under accession number DSM 25840.
6. Use according to any preceding paragraph, wherein the at least two Bacillus strains comprise or consist of the Bacillus subtilis strain deposited at DSMZ under accession number DSM 32324, the Bacillus amyloliquefaciens strain deposited at DSMZ under accession number DSM 25840 and the Bacillus subtilis strain deposited at DSMZ under accession number DSM 32325.
7. Use according to any preceding paragraph, wherein the at least two Bacillus strains are present as spores.
8. Use according to any preceding paragraph, wherein the composition is an animal feed additive.
9. Use according to any preceding paragraph, wherein the composition is in the form of a dry powder, a capsule or an oral paste.
10. Use according to any preceding paragraph, wherein the composition comprises one or more animal feed, animal feed additive or animal feed premix ingredients selected from the group consisting of vitamins, minerals, enzymes, amino acids, flavours, yeast extract, active charcoal, mineral clay, silica or a combination thereof.
11. A method for feeding a pet animal, such as a dog or a cat, comprising administering to the animal an animal feed, animal feed additive or animal feed premix composition comprising at least two Bacillus strains selected from:
   i. the Bacillus subtilis strain deposited at Deutsche Sammlung von Microorganismen und Zellkulturen GmbH (DSMZ) under accession number DSM 32324,
   ii. the Bacillus subtilis strain deposited at DSMZ under accession number DSM 32325, and
   iii. the Bacillus amyloliquefaciens strain deposited at DSMZ under accession number DSM 25840.
12. A method for improving gastrointestinal health and faecal score in a pet animal, such as a dog or a cat, the method comprising administering to the animal an effective amount of an animal feed, animal feed additive or animal feed premix composition comprising at least two Bacillus strains selected from:
   i. the Bacillus subtilis strain deposited at Deutsche Sammlung von Microorganismen und Zellkulturen GmbH (DSMZ) under accession number DSM 32324,
   ii. the Bacillus subtilis strain deposited at DSMZ under accession number DSM 32325, and
   iii. the Bacillus amyloliquefaciens strain deposited at DSMZ under accession number DSM 25840.
13. A method for improving the uptake of energy and protein in an animal, wherein the animal is a pet animal such as a dog or a cat, the method comprising feeding the animal with an animal feed, animal feed additive or animal feed premix composition comprising at least two Bacillus strains, wherein the Bacillus strains are selected from:
   (a) the Bacillus subtilis strain deposited at Deutsche Sammlung von Mikroorganismen und Zellkulturen GmbH (DSMZ) under accession number DSM 32324, and
   (b) the Bacillus subtilis strain deposited at DSMZ under accession number DSM 32325, and
   (c) the Bacillus amyloliquefaciens strain deposited at DSMZ under accession number DSM 25840.
14. The method of any of paragraphs 11-13, wherein the animal feed, animal feed additive or animal feed premix composition comprises from 10⁵ to 10¹² colony forming units per gram (CFU/g) of each Bacillus strain in the composition.
15. The method of any of paragraphs 11-14, wherein the animal is a dog.
16. An animal feed, animal feed additive, or animal feed premix composition comprising:
   a. from 10⁵ to 10¹² colony forming units per gram (CFU/g) of at least two Bacillus strains selected from:
      i. the Bacillus subtilis strain deposited at Deutsche Sammlung von Microorganismen und Zellkulturen GmbH (DSMZ) under accession number DSM 32324,
      ii. the Bacillus subtilis strain deposited at DSMZ under accession number DSM 32325,
      iii. the Bacillus amyloliquefaciens strain deposited at DSMZ under accession number DSM 25840; and
   b. one or more animal feed, animal feed additive, or animal feed premix ingredients wherein the at least two Bacillus strains is effective to improve gastrointestinal health and faecal score in the animal receiving the animal feed, animal feed additive, or animal feed premix.
17. The composition of paragraph 16, wherein the at least two Bacillus strains comprises a Bacillus subtilis strain and a Bacillus amyloliquefaciens strain.
18. The composition according to paragraph 16 or 17, wherein the at least two Bacillus strains comprises the Bacillus subtilis strain deposited at DSMZ under accession number DSM 32324 and the Bacillus amyloliquefaciens strain deposited at DSMZ under accession number DSM 25840.
19. The composition according to any of paragraphs 16-18, wherein the at least two Bacillus strains consists of the Bacillus subtilis strain deposited at DSMZ under accession number DSM 32324, the Bacillus amyloliquefaciens strain deposited at DSMZ under accession number DSM 25840 and the Bacillus subtilis strain deposited at DSMZ under accession number DSM 32325.
20. The composition according to any of paragraphs 16-19, wherein the at least two bacillus strains are present as spores.
21. The composition according to any of paragraphs 16-20, wherein the composition is an animal feed additive.
22. The composition according to any of paragraphs 16-21, wherein the composition is in the form of a dry powder, a capsule or an oral paste.
23. The composition according to any of paragraphs 16-22, wherein the animal feed, animal feed additive, or animal feed premix ingredients is selected from the list including vitamin(s), mineral(s), enzyme(s), amino acid(s), flavours, yeast extract, active charcoal, mineral clay, silica or a combination thereof.
24. A method for feeding an animal comprising administering an animal feed, animal feed additive, or animal feed premix composition comprising at least two Bacillus strains selected from:
   i. the Bacillus subtilis strain deposited at Deutsche Sammlung von Microorganismen und Zellkulturen GmbH (DSMZ) under accession number DSM 32324,
   ii. the Bacillus subtilis strain deposited at DSMZ under accession number DSM 32325, and
   iii. the Bacillus amyloliquefaciens strain deposited at DSMZ under accession number DSM 25840.
25. The method of paragraph 24, wherein the at least two Bacillus strains are administered in an amount effective to improve gastrointestinal health and faecal score in the animal.
26. The method according to paragraph 24 or 25, wherein the animal is a pet animal, such as a dog or cat.
27. A method for improving gastrointestinal health and faecal score in an animal, the method comprising administering an effective amount of a composition according to any of paragraphs 16-23 to an animal in need thereof.
28. A method of increasing release of sugars and protein solubility of an animal feed, comprising adding a composition comprising at least two Bacillus strains to an animal feed, wherein the Bacillus strains are selected from:
   (a) the strain deposited at Deutsche Sammlung von Mikroorganismen und Zellkulturen GmbH (DSMZ) under accession number DSM 32324, and
   (b) the strain deposited at DSMZ under accession number DSM 32325, and
   (c) the strain deposited at DSMZ under accession number DSM 25840

### EXAMPLES

### Example 1

An in vitro study was conducted to investigate the ability of the composition of the present disclosure comprising at least two Bacillus strains to release sugars from commercially available dry dog kibble, thereby obtaining a measurement of degradation of the carbohydrate portion in the dry dog kibble.

Samples of commercially available dog kibble diets were tested with and without the addition of the composition of the present disclosure comprising at least two Bacillus strains.

The method principle is that animal feed samples are mixed in liquid media with the composition of the disclosure (probiotics sample denoted “PRO”) and without the composition of the invention (control sample denoted “Control”) and, following growth at standardized conditions, supernatants are collected. Supernatants are then mixed with the yellow compound 3.5-dinitrosalicylic acid (DNS), which in the presence of simple sugars (also called reducing sugars, because they act as reducing agents) will turn to the darker colored red/brown compound 3-amino-5-nitrosalicylic acid. By using a spectrophotometer to measure the intensity of the dark color, the amount of released reducing sugars is determined, and thus the capacity of the composition of the disclosure to break down carbohydrates into simple sugars.

The composition comprising probiotic strains belonging to the Bacillus genus, which is based on viable spores of Bacillus amyloliquefaciens and Bacillus subtilis (PRO sample) caused increased reducing sugar release from the commercially available dog kibble compared to the untreated control (control sample), irrespective of the amount of protein and fiber in the dog kibble (labeled as “glucose equivalents” in FIG. 1). An additional 620 or 501 kJ glucose/kg were released from the two respective kibble diets. Without being bound by theory it is believed that an increase in release of sugars is an advantage as it is a measurement of the amount of energy released to the dog upon ingestion of the dog kibble.

### Example 2

Proteins are complex molecules present in all living organisms and provide many essential functions in the body. These molecules are formed through the ‘building blocks’ of 20 amino acids, which can be deemed essential or non-essential—those deemed essential cannot be synthesized in the body and must be obtained through dietary intake, whereas non-essential can be synthesized by the body through the metabolism of other amino acids. The number of essential amino acids depends on the species in question. Solubilization of protein is thus needed for supplying essential amino acids to build hair, skin, nails, muscles, tendons, ligaments, and cartilage. Protein also plays a main role in hormone production and promotes immune system function.

An in vitro assay was performed that allows to investigate the impact of the composition of the present disclosure on solubility of protein. The physical structure of protein can have an impact on its digestibility. By exposing proteins to proteases and other protein-degrading enzymes, you can change their structure and improve their digestibility.

Samples of commercially available dog kibble diets were tested with and without the addition of the composition of the present disclosure comprising at least two Bacillus strains.

The method principle is that animal feed samples are mixed in liquid media with the composition of the disclosure (probiotics sample denoted “PRO”) or without the composition of the invention (control sample denoted “Control”) and following growth at standardized conditions the supernatants are collected. The supernatants are then mixed with culpric sulphate and bicinchoninic acid (BCA). The solubilized proteins will reduce the Cu2+ from cupric sulphate to Cu1+ which then reacts with the bicinchoninic acid (BCA) resulting in an intense purple-colored reaction product which exhibits a strong linear absorbance at 562 nm with increasing protein concentrations. Thus, the more solubilized protein generated from the feed sample by the probiotic, the stronger the color reaction.

The composition comprising probiotic strains belonging to the Bacillus genus, which is based on viable spores of Bacillus amyloliquefaciens and Bacillus subtilis (PRO sample) caused increased protein solubility compared to the untreated control (control sample), irrespective of the amount of protein and fiber in the dog kibble (labeled as “g of protein” in FIG. 2). An additional 14 or 13 g of protein/kg of respective kibble diets were made available from the two respective kibble diets. Without being bound by theory it is believed that an increase in protein solubility is an advantage as it is a measurement of the digestibility of the kibble and thus the release of amino acids to the dog upon ingestion of the dog kibble.

### Example 3

An in vivo study with dogs was conducted in Spain. The objective of this study was to evaluate the effect of a composition comprising probiotic strains belonging to the Bacillus genus, which is based on viable spores of Bacillus amyloliquefaciens and Bacillus subtilis. Without being bound by theory the hypothesis was that the probiotic will positively impact dry matter content in faeces and faecal score.

A total of 40 healthy dogs were randomly distributed in 2 groups of 20 dogs balanced for age, sex and body weight. Before trial start the dogs received only a control diet (complete feed) for a 21 days adaptation period. After the adaptation period, the two groups of dogs received Control diet (T1) or Control diet supplied with the composition comprising the probiotic strains (T2) for 35 days.

The dogs were group-housed but fed individually. The dogs were fed with a complete dry extruded diet and the probiotic or placebo were applied as top-dressing once daily corresponding to a target dose of 1.0×10⁹ cfu/kg food.

Dogs were weighed weekly and the amount of food offered to them was increased or decreased if their body weight decreased or increased by more than 5% initial body weight. Faecal samples were taken on days 0, 7, 28 and 35. Feed intake and faecal score were performed daily. Daily faecal consistency was determined by faecal score for each dog. Stool was graded on a scale ranging from 1 (hard dry stool) to 5 (liquid stool) according to consistency, a faecal score of 2.5 being considered optimal (firm, fully formed stool), see detailed scoring scale in Table 2.

Table 3 reports the main outcome of the study. It can be concluded that a significant reduction in the number of days with unacceptable scores (>score 3) was observed in the group of dogs fed the probiotic (P=0.041). Furthermore, it can be concluded that the dry matter content in faeces at study end day 35 was increased in the group of dogs fed the probiotic (P=0.054). It is further interesting to note that the significant improved effect observed on the faecal score is not only a result of an increase in Dry Matter Content. For several of the measurements the Dry Matter Content is lower in the probiotic group than the control group, however without affecting the overall faecal score improvement. This indicates that the overall gastrointestinal health of the animal is improved.

TABLE 2
Fecal scoring scale:

| Score | Description 1 |
|---|---|
| 1.0 | Hard dry and crumbly; “Bullet-like” |
| 1.5 | Hard and dry |
| 2.0 | Well formed; does not leave a mark |
| | when picked up; “kickable” |
| 2.5 | Well formed, with a slightly moist |
| | surface, which leaves a mark when picked up; |
| | almost sticky to touch |
| 3.0 | Moist beginning to lose form, |
| | leaving a definite mark when picked up |
| 3.5 | Very moist, but still has some definite form |
| 4.0 | The majority, if not all the form is lost; |
| | poor consistency; viscous |
| 4.5 | Diarrhoea, with some areas of consistency |
| 5.0 | Watery diarrhoea |

TABLE 3

| | T1 Control | T2 Probiotic | P-value |
|---|---|---|---|
| Body weight, kg | | | |
| Day 0 | 12.7 | 12.8 | 0.758 |
| Day 35 | 12.1 | 12.3 | 0.788 |
| Dry matter in | | | |
| feces, % | | | |
| Day 0 | 37.0 | 32.2 | 0.295 |
| Day 7 | 32.1 | 31.2 | 0.548 |
| Day 28 | 33.6 | 33.4 | 0.913 |
| Day 35 | 32.8 | 35.0 | 0.054 |
| Faecal score: | | | |
| Total number of days | 0.80 | 0.30 | 0.041 |
| per dog with score >3 | | | |

The present invention has been described with reference to various embodiments, aspects, examples, or the like. It is not intended that these elements be read in isolation from one another. Thus, the present disclosure provides for the combination of two or more of the embodiments, aspects, examples, or the like.

All embodiments described herein are intended to be within the scope of the invention disclosed. These and other embodiments of the present invention will become readily apparent to those skilled in the art from the following detailed description of the preferred embodiments having reference to the whole description, the invention not being limited to any particular preferred embodiment(s) disclosed.

1-15. (canceled)

16. A method comprising feeding to a dog at least two Bacillus strains selected from:
i. the Bacillus subtilis strain deposited at Deutsche Sammlung von Microorganismen und Zellkulturen GmbH (DSMZ) under accession number DSM 32324,
ii. the Bacillus subtilis strain deposited at DSMZ under accession number DSM 32325, and
iii. the Bacillus amyloliquefaciens strain deposited at DSMZ under accession number DSM 25840;
in amounts sufficient to improve a faecal score in said dog.

17. The method of claim 16, wherein said at least two Bacillus strains comprises the Bacillus subtilis strain deposited at DSMZ under accession number DSM 32324 and the Bacillus subtilis strain deposited at DSMZ under accession number DSM 32325.

18. The method of claim 16, wherein said at least two Bacillus strains comprises the Bacillus subtilis strain deposited at DSMZ under accession number DSM 32324 and the Bacillus amyloliquefaciens strain deposited at DSMZ under accession number DSM 25840.

19. The method of claim 16, wherein said at least two Bacillus strains comprises the Bacillus subtilis strain deposited at DSMZ under accession number DSM 32325 and the Bacillus amyloliquefaciens strain deposited at DSMZ under accession number DSM 25840.

20. The method of claim 16, wherein said at least two Bacillus strains comprises the Bacillus subtilis strain deposited at DSMZ under accession number DSM 32324 and the Bacillus subtilis strain deposited at DSMZ under accession number DSM 32325 and the Bacillus amyloliquefaciens strain deposited at DSMZ under accession number DSM 25840.

21. The method of claim 16, wherein said at least two Bacillus strains are fed to said dog as part of a composition comprising 10⁵ to 10¹² colony forming units of said at least two Bacillus strains per gram of said composition.

22. The method of claim 16, wherein said at least two Bacillus strains are fed to said dog as part of a composition comprising 10⁸ to 10¹¹ colony forming units of said at least two Bacillus strains per grain of said composition.

23. The method of claim 16, wherein said at least two Bacillus strains are fed to said dog as part of a composition comprising 10⁹ to 10¹⁰ colony forming units of said at least two Bacillus strains per gram of said composition.

24. The method of claim 16, wherein said at least two Bacillus strains are fed to said dog as part of a composition comprising at least 5×10⁴ colony forming units of each of said at least two Bacillus strains per gram of said composition.

25. The method of claim 16, wherein said at least two Bacillus strains are fed to said dog as part of a composition that is formulated as a dry powder or capsule.

26. The method of claim 16, wherein said at least two Bacillus strains are fed to said dog as part of a composition that is formulated as an oral paste.

27. An animal feed additive comprising at least two Bacillus strains selected from:
i. the Bacillus subtilis strain deposited at Deutsche Sammlung von Microorganismen und Zellkulturen GmbH (DSMZ) under accession number DSM 32324,
ii. the Bacillus subtilis strain deposited at DSMZ under accession number DSM 32325, and
iii. the Bacillus amyloliquefaciens strain deposited at DSMZ under accession number DSM 25840;
in amounts sufficient to improve an average faecal score of dogs fed said animal feed additive.

28. The animal feed additive of claim 27, wherein said at least two Bacillus strains comprises the Bacillus subtilis strain deposited at DSMZ under accession number DSM 32324 and the Bacillus subtilis strain deposited at DSMZ under accession number DSM 32325.

29. The animal feed additive of claim 27, wherein said at least two Bacillus strains comprises the Bacillus subtilis strain deposited at DSMZ under accession number DSM 32324 and the Bacillus amyloliquefaciens strain deposited at DSMZ under accession number DSM 25840.

30. The animal feed additive of claim 27, wherein said at least two Bacillus strains comprises the Bacillus subtilis strain deposited at DSMZ under accession number DSM 32325 and the Bacillus amyloliquefaciens strain deposited at DSMZ under accession number DSM 25840.

31. The animal feed additive of claim 27, wherein said at least two Bacillus strains comprises the Bacillus subtilis strain deposited at DSMZ under accession number DSM 32324 and the Bacillus subtilis strain deposited at DSMZ under accession number DSM 32325 and the Bacillus amyloliquefaciens strain deposited at DSMZ under accession number DSM 25840.

32. The animal feed additive of claim 27, comprising 10⁵ to 10¹² colony forming units of said at least two Bacillus strains per gram of said animal feed additive.

33. The animal feed additive of claim 27, comprising 10⁸ to 10¹¹ colony forming units of said at least two Bacillus strains per gram of said animal feed additive.

34. The animal feed additive of claim 27, comprising 10⁹ to 10¹⁰ colony forming units of said at least two Bacillus strains per gram of said animal feed additive.

35. The animal feed additive of claim 27, comprising at least 5×10⁴ colony forming units of each of said at least two Bacillus strains per gram of said animal feed additive.
